(19) United States
(12) Patent Application Publication
(10) Pub. No.: US 2024/0245809 A1
(43) Pub. Date: Jul. 25, 2024

(54) A CONTRAST AGENT FOR MRI IMAGING DIAGNOSTICS

(71) Applicant: Catharina Philippina Janssen, Molenhoek (NL)

(72) Inventors: Catharina Philippina Janssen, Molenhoek (NL); Jelle Onno Barentsz, Molenhoek (NL); James Nagarajah, Molenhoek (NL); Vasant Kumar, Molenhoek (NL); Rumen Tomov, Molenhoek (NL)

(73) Assignee: Catharina Philippina Janssen, Molenhoek (NL)

(21) Appl. No.: 18/573,534

(22) PCT Filed: Jun. 23, 2022
(86) PCT No.: PCT/EP2022/067255

§ 371 Date: Dec. 22, 2023

(30) Foreign Application Priority Data
Jun. 25, 2021 (NL) 2028542

(51) Int. Cl. A61K 49/18

(52) U.S. Cl. CPC A61K 49/1854

(57) ABSTRACT

A contrast agent for MRI imaging diagnostics. The contrast agent is comprised of a nano-fuel cell coupled to at least one ligand for specific binding to a cell membrane. An imaging method for the visualisation of tumor cells using the contrast agent and a method for non invasive treatment of tumor cells using the contrast agent.

The present invention relates to a contrast agent for MRI imaging diagnostics. The contrast agent is comprised of a nano-fuel cell coupled to at least one ligand for specific binding to a cell membrane. The present invention further relates to an imaging method for the visualisation of tumor cells using the contrast agent and a method for non-invasive treatment of tumor cells using the contrast agent.

Tumor targeting is a rapidly expanding technique applied for cancer treatment as well as use thereof in the visualization of tumors. Targeted anticancer therapies consist generally of antibodies or antibody-derived fragments, proteins, peptides, small molecule inhibitors, or DNA/RNA aptamers directing an attached drug to the tumor cell. Tumor targets are in general membrane proteins or “tracers” with enhanced expression on tumor or tumor-associated cells, such as malignant cells or angiogenic endothelial cells. These membrane proteins with enhanced expression on tumor or tumor-associated cells are especially suited for the development of tumor-visualizing compounds or contrast agents for medical imaging diagnostics, which could for example be used for the early diagnosis or localization of tumors, i.e. tumor-targeted imaging.

For example, for the detection and to determine the disease progression of prostate cancer a targeted treatment is provided based on the highest probability of a cure with the least of side effects. To determine the cancer and treatment tracers (membrane proteins), which specifically bind to the prostate tumor are being used. An example is the PSMA (prostate specific membrane antigen), which is used in the PET/CT scans for visualisation of the prostate cancer. A radioactive isotope (for example 18F, 68Ga) is linked to the PSMA traces to observe the location and size of the tumor.

There are over 10.000 potential membrane proteins that may be of use in medical visualization diagnostics. However, there is still little knowledge about which targets should be used for optimal results per tumor type, or even better, per individual tumor or patient. One of the drawbacks is that the membrane proteins which are very suitable for CT and PET imaging technologies, such as the PSMA tracer for prostate cancer, are unsuitable for use in MRI imaging, because the usual MRI contrast agents are not strong enough to get associated with these membrane proteins, such as PSMA, to be observed in MRI imaging. At present a laborious and expensive (>4 Mil) PET-MRI apparatus and method is required for visualisation and diagnostics to such as PSMA.

Considering the above, there is a need in the art for an improved more versatile contrast agent for use in medical imaging diagnostics, more specifically MRI imaging. In addition, there is a need in the art for an improved MRI imaging method for the visualisation of tumor cells.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a contrast agent for MRI imaging diagnostics comprised of a nano fuel cell coupled to at least one ligand for specific binding to a cell membrane, wherein the nano fuel cell is comprised of an anode compartment including an anode, a cathode compartment including a cathode, and a suspension for generating a current of electrons, wherein said suspension is preferably comprised of a plurality of hollow particles in electrically conductive contact, wherein said hollow particles comprise entrapped therein a redox-reaction for catalyzing an enzymatic conversion of a substrate in said hollow particles thereby liberating electrons, wherein said hollow particles comprise a substrate permeable and electrically conductive outer polymer shell, and said suspension is disposed within said anode compartment, or within said cathode compartment, or between said anode and cathode compartment. The nanofuel cell is comprised of an anode and a cathode nano-electrodes delivered to the tumor in the form of a suspension (nano-inks). The electrodes, anode and cathode, may comprise electronically conductive nano-carriers (e.g. carbon nano tubes CNTs or gold nanoparticles nAu) coated with redox selective enzyme catalyst immobilised on the carrier surface with or without mediators. Additionally, both nano electrodes are conjugated with one or more ligands selected from the group consisting of affinity binding molecules (antibodies, mini-bodies, FAB fragments, ligand proteins, peptides, RNAs, small molecules and nano-particles) in order to ensure selective electron conducting bonding to the tumor specific surface.

The contrast agent of present invention comprises a nano fuel cell that is coupled or bonded to a ligand or tracer of the cell membrane of the tissue or cell to be detected by MRI imaging technology. In contrast to the ligand alone, which is not detectable via MRI, nano fuel cells coupled to the ligands are visible using MRI, because the fuel cells selectively localized onto the tumor surface discharge a heartbeat rate modulated current through the tumor membrane/tissue. Such current generates sufficiently strong localized modulated magnetic field, which locally disrupts the magnetic field within the MRI and therefore becomes visible. For example, with the nano fuel cell coupled to PSMA, it is possible to observe and monitor on the MRI the PSMA thereby visualize the prostate cancer. The tumor cells are visualized due to the accumulation of the nano fuel cells at the tumor cell locations, because the ligand linked to the fuel cell directs and binds to the cell membrane target of the tumor cell. Disturbances in the magnetic field due to the electrical current emitted by the fuel cells provide the visualisation during the MRI scan and enables visualisation of the specific tumor.

The nano fuel cell of the contrast agent uses naturally occurring in the human body biofuel, like glucose, and can deliver a current modulated with a frequency of the fuel delivery (e.g. heartbeat rate). The contrast agent of present invention comprising the nano fuel cell using glucose is also referred to herein as Enzymatic Glucose fuel cell (EGFC). Since it can been designed in such a way that the components of such a battery are not toxic/biocompatible it is safe for use in in vivo environment (for example a pace maker in humans). In difference to the conventional methods (for example using radioactive ligands in the PET-CT) to visualize the cancer cells, the present contrast agent does not need to use radioactive materials, e.g. easier and more safe to use, is fully biodegradable and can be used in the MRI, which provides a much sharper and more contrast image, as compared to PET-CT for the detection of tumor cells.

The fuel cell of the contrast agent comprises a suspension that can be used to generate an electron flow, the suspension comprising a polypeptide, characterized in that the polypeptide is enclosed in a hollow particle, the hollow particle is not permeable to the polypeptide. Because the polypeptide is entrapped and not embedded in the shell of the hollow particle, the hollow particle could have a shell that has unique properties, such as selective permeability, robustness and conductivity. There is no accumulation of electrons inside the hollow particles and transport of electrons is proceeding via the outer shell. The hollow particle preferably contains an enzyme, preferably the enzyme glucose oxidase, that can convert glucose into gluconolactone, releasing electrons. There are many enzymes known that catalyse a chemical reaction that release electron(s) and that can be used in the contrast agent of present invention. Therefore, this bio-battery can be used in imaging diagnostics when coupled to a ligand and provide improved diagnostic imaging for example in MRI.

According to a preferred embodiment, the present invention relates to the contrast agent, wherein the at least one ligand is one or more selected from the group consisting of affinity binding molecules, antibodies, mini-bodies, FAB fragments, ligand proteins, peptides, RNAs, small molecules and nanoparticles. Tumors can be targeted with an extensive arsenal of monoclonal antibodies, ligand proteins, peptides, RNAs, and small molecules. In addition to therapeutic targeting, some of these compounds can also be applied for tumor visualization before or during surgery, after conjugation with radionuclides and/or near-infrared fluorescent dyes. Most of these tumor-targeting compounds are directed against cell membrane-bound proteins. Various categories of targetable membrane-bound proteins, such as anchoring proteins, receptors, enzymes, and transporter proteins, exist.

According to another preferred embodiment, the present invention relates to the contrast agent, wherein the at least one ligand is able to bind to a specific cell membrane protein, selected from the group consisting of a tumor cell membrane protein, PSMA, FAP, CAIX, SSTR, αvβ3 integrin, Bombesin R, CEA, CD13, CD44, CXCR4, EGFR, ErbB-2, Her2, Emmprin, Endoglin, EpCAM, EphA2, Folate, GRP78, IGF, Matriptase, Mesothelin, cMET/HGFR, MT1-MMP, MT6-MMP, Muc-1, PSCA, Tn antigen and uPAR, preferably PSMA, FAP, CAIX, and/or SSTR. In general most membrane proteins that are overexpressed on tumor or tumor-associated cells are potentially suitable for tumor-targeted imaging and can be used for selection of suitable ligands to be used in the contrast agent of present invention.

According to yet another preferred embodiment, the present invention relates to the contrast agent, wherein the at least one ligand is furthermore conjugated with radionuclides and/or near-infrared fluorescent dyes selected from the group consisting of beta particles emitting isotopes, alpha particles emitting isotopes, auger electrons emitting isotopes, fluorophores, F-18, Ga-68, Zr-89, Cu64, I-124, Tc-99m, In-111, and I-123. When the ligand is further conjugated with for example a radioactive isotope or fluorophore, the MRI contrast agent is suitable for use in for example PET and SPECT imaging technology and use in photodynamic therapy and imaging purposes.

According to yet another preferred embodiment, the present invention relates to the contrast agent, wherein the polymer of said outer polymer shell is a block-copolymer comprising a hydrophobic polystyrene and a hydrophilic polyisocyanopeptide. The hollow particle is a vesicle, preferably a polymersome composed of amphiphilic molecules that form the outer shell. A polymersome is a vesicle that is constructed from polymeric amphiphilic building blocks and can have unique properties such as rigidity and the ability to conduct electrons. The hollow particle is composed of conductive polymer that can easily conduct electrons, preferably a block-copolymer. Preferably the block-copolymer comprises polystyrene-b-poly (L-isocyanoalanine (2-thiophen-3-yl-ethyl)amide) (PS-PIAT). And preferably the side groups present on the block-copolymer are polymerized, more preferably the thiophene side groups present in the side chain of polystyrene-b-poly (L-isocyanoalanine (2-thiophen-3-yl-ethyl)amide) are polymerized. PS-PIAT is able to form very stable and well-defined polymersomes in water and enzymes can be successfully incorporated within the aqueous inner compartment of the vesicles composed of PS-PIAT.

According to another preferred embodiment, the present invention relates to the contrast agent, wherein the redox-reaction catalyzing enzyme is glucose oxidase (GOx) and the substrate is glucose, preferably GOx in combination with horseradish peroxidase (HRP). Preferable GOx and HRP are present at the cathode. The electrochemical reaction is the conversion of ss-D-glucose into D-glucono-1,5-lactone by GOx. During this reaction two electrons are released that are transported through the nano-carrier conductive body (e.g. functionalized carbin nanotube or Au nanoparticle) or shell of the hollow particle, and through the conductive outer shell and/or conjugating ligand. A particular problem for the in vivo application of the nano fuel cells comprised of bio-cathodes and -anodes is the low concentration of dissolved oxygen in the blood. Free O₂ concentration is almost five times lower in blood and ten times lower in saliva, compared to air-saturated ex vivo electrolytes. Most of the oxygen molecules are bound to the haemoglobin in the human vascular system or consumed during different enzymatic reactions. Oxidases (including laccases and bilirubin oxidases) are conventionally utilized in enzymatic cathodes due to their ability to achieve electrocatalytic reduction of oxygen at high onset potentials. Adversely, their use in in vivo applications is limited by the necessary acidic optimal value of the pH. Additionally, such bio-cathodes suffer from inhibition by any increase of H₂O₂ concentration, as well as instability caused by deactivation via inhibitors like chloride ions. To overcome this issue the contrast agent of present invention that is suitable for the in-vivo application may be comprised of a co-immobilization of glucose oxidase (GOx) and horseradish peroxidase (HRP) on cathode electrode surfaces (for example CNT surfaces). In this case, the GOx produces H₂O₂ from the oxidation of glucose, and HRP achieves the electrocatalytic reduction of H₂O₂ into water at a low over potential via Direct Electron Transfer (DET). Such bi-enzymatic cathode design may alleviate the issue related to enzymatic cathode instability in an in vivo oxygen deficient environment. As such, the choice of enzymes and their immobilization in EGFCs presents an important challenge in the design of the nano-fuel cells for use in the contrast agent of present invention in terms of selectivity for oxygen reduction and glucose oxidation that circumvents the need for a membrane separation. The design of the bio-anodes is focused on the use of glucose oxidase (GOx), while a bi-enzymatic approach is adopted for the bio-cathodes based on the combination of GOx and horseradish peroxidase (HRP). GOx was used as bio-anode using mediated electron transfer via 1,4-naphthoquinone (NQ). Both the cathode and the anode may utilise various carbon nanotube forms (multiwall-walled carbon nanotubes MWCNT or single wall carbon nanotubes SWCNTs) as immobilization vehicles for optimal coupling of ligands.

According to another preferred embodiment, the present invention relates to the contrast agent, wherein the cathode and/or anode are enzyme-coated with redox-reaction catalyzing enzyme(s), preferably the cathode and/or anode are coated with glucose oxidase (GOx), more preferably GOx in combination with horseradish peroxidase (HRP).

According to yet another preferred embodiment, the present invention relates to the contrast agent, wherein the nano fuel cell further comprises Naphthoquinone (NQ) as mediator at the anode electrode or wherein the nano fuel cell further comprises amino acids, preferably histamine. A direct electronic transfer (DET) between the catalyst and the carrier is often hindered because the enzymes have their catalytic sites buried within the protein matrix, which can insulate the redox site and eventually prevent the DET. In DET the cell voltage can be reduced due to over potentials caused by a hindered charge transfer, mass transport or resistive losses. Alleviating the 35 issue, one could consider utilization of a mediated electron transfer (MET) mechanism. However, some mediators present health hazards or are susceptible to chemical deterioration in an in-vivo environment. In addition, in MET-based systems a certain potential difference between the enzyme and the mediator has to be accounted for, which commonly leads to a loss of cell voltage. By using Naphthoquinone (NQ) the hurdle is overcome as NQ is known to provide an efficient mediated oxidation of glucose with almost an order of magnitude increase in catalytic current densities. 1,4-Naphthoquinone or para-naphthoquinone is a natural organic compound derived from naphthalene. 1,4-Naphthoquinones are common metabolites of plants, animals, fungi and bacteria. An additional advantage of 1,4-Naphthoquinones is their biocompatibility. Furthermore, amino acids, like histamine can be used in combination with the nano fuel cell since histamine acts as a neurotransmitter for at least the brain and/or spinal cord and binding agent to these cells, thereby further improving and strengthening output signal of the contrast agent for use in MRI diagnostics.

According to a preferred embodiment, the present invention relates to the contrast agent, wherein the hollow particles are embedded in an electrically conductive matrix comprised of ferrocene and/or viologen derivatives. The suspension and/or nano electrodes may comprise electron carriers such as ferrocene derivatives and viologen derivatives in order to facilitate electron transport.

According to yet another preferred embodiment, the present invention relates to the contrast agent, wherein the contrast agent is an MRI imaging contrast agent. The contrast agent is furthermore suitable for medical imaging diagnostics selected from the group consisting of CT, PET and SPECT.

According to yet another preferred embodiment, the present invention relates to the contrast agent, wherein said nano-fuel cell coupled to at least one ligand has a particle size of approximately 20 to 400 nm, preferably 50 to 250 nm, more preferably 75 to 200 nm. If the particle size is smaller than 20 nm, the liver of the patient will remove the contrast agent too quickly from the patient to be an affective contrast agent.

According to yet another preferred embodiment, the present invention relates to the contrast agent, wherein the cathode and the anode are comprised of carbon, preferably single or multiwall-walled carbon nanotubes (SWCNT or MWCNT), and/or metal nanoparticles, preferably gold nanoparticles, for coupling of the at least one ligand to the nano-fuel cell. The contrast agent of present invention is comprised of a nano-fuel cell that needs to provide an electronic transfer from the catalyst to the load to ensure that the expected redox reaction is taking place. Therefore, carbon (e.g. carbon nanotubes—CNTs) and/or metal (e.g. gold) nano-formations on the cathode and anode are most suitable candidates to ensure an optimal electronic transfer. Carbon nanotubes (CNTs), including multiwall-walled carbon nanotubes (MWCNT) may be a preferred material due to their high specific surface and high conductivity, as well as for their Direct Electron Transfer (DET) properties towards some types of redox enzymes.

According to a preferred embodiment, the present invention relates to the contrast agent, wherein the at least one ligand comprises pyrenyl residues for stable non-covalent coupling to the nano-fuel cell, preferably coupling with the multiwall-walled carbon nanotubes (MWCNT). Both the cathode and the anode are preferably comprised of multiwall-walled carbon nanotubes (MWCNTs) as immobilization electronically conductive vehicles. To attach the ligand that is able to bind to a specific cell membrane protein, including pharmacophores like PSMA, FAP and sstr2 inhibitors to MWCNTs they are preferably derivatized with a pyrenyl residue, which form exceptionally stable non-covalent bonds with carbon nanotubes. This could be achieved, e.g. using active esters of pyrenyl butyric acid as a derivatizing reagent to enable and ensure that on the one hand the ligand or pharmacophore group is bound with sufficient strength but is still able to have strong target binding function, and on the other side this strong target should not hinder or hamper an attachment of the appropriate enzymes to nanoelectrodes. To further improve these binding affinities the application of composite carbon/gold nanocarriers may be used. In this case orthogonal conjugation methods, e.g., Au-selective derivatization with lipoic acid-modified pharmacophores, or first thiol derivatization of Au nanocomponent followed by the selective attachment of, e.g., maleimide substituted pharmacophores and, thereafter, the conjugation of enzymes to the nanocarbon part of the composite could be used for the decoration of the multiwall-walled carbon nanotubes.

According to another preferred embodiment, the present invention relates to the contrast agent, wherein the outer polymer shell is coated with metal particles selected from the group consisting of Ag, Au, Pt, Ti, preferably Au. The outer shell of the contrast agent, or more specifically the nano-fuel cell, may be coated with metal particles for example by surface modification using gold (Au) particles (e.g. by producing thiol-capped Au nanoparticles), to further improve a selective binding of the contrast agent comprising the nano-fuel cell on the tumor cell surface.

The present invention, according to a second aspect, relates to an imaging method for the visualisation of tumor cells comprising the steps of;
a) providing a contrast agent of present invention to a patient,
b) providing glucose to said patient for providing activation of the contrast agent and generating a current of electrons that is detectable by medical imaging techniques,
c) obtaining an image of the contrast agent using medical imaging techniques.

According to yet another preferred embodiment, the present invention relates to the method, wherein the medical imaging technique selected from the group consisting of MRI, Magnetometric imaging (MEG), CT, PET and SPECT, preferably MRI.

According to another preferred embodiment, the present invention relates to the method, wherein said contrast agent is detectable by imaging diagnostics for 1 to 90 minutes, preferably 5 to 75 minutes, more preferably 10 to 60 minutes, after providing said contrast agent to said patient. After administrating the contrast agent, the substrate, i.e. glucose, will go trough the permeable and electrically conductive outer polymer shell and/or be delivered to both electrodes of the nano fuel cell, thereby initiating enzymatic reactions and release of electrons through the load presented by the tumor which can be detected by MRI. This reaction and detection can be maintained and monitored for at most 90 minutes with MRI, after which the signal gradually fades.

The present invention, according to a further aspect, relates to a method for non-invasive treatment of tumor cells or macrophages comprising the steps of;
a) providing a contrast agent of present invention to a patient,
b) providing an overdose of glucose to said patient resulting in electrocution and/or electroporation of the tumor cells or macrophages.
   Next to imaging purposes, the contrast agent can be used in tumor destruction by electrophoration or electrocution of the tumor. Since the fuel cell emits an electrical current at a specific location, it may also provide the possibility to emit a stronger electrical current to enable electrocution or ablation of the tumor cell and site specific destruction (i.e. at the cell surface of a tumor cell). At present, in so-called IRE (Irreversible Electoporation) tumor cells are electrocuted where a few needles are inserted around prostate cancer and then the tumor is selectively destroyed. A similar method is possible using the contrast agent of present invention wherein the nano fuel cell is coupled to the ligand for specific binding to a cell membrane or macrophages, such as PSMA, and electrocute the prostate tumor or macrophages in this way. The overdose of glucose added will such that the amount of glucose provided will be close to the maximum glucose levels of the patient for maximum electron generation by the contrast agent of present invention. In addition to increasing the amount of glucose, and external energy application (e.g. radio-wave, electric current like that of an AID, ultrasound wave) may “charge” the nano fuel cells and induce an instant discharge. The nano fuel cell then acts like a ‘conductor’. The method of present invention for the destruction of prostate tumor cells can be compared with the existing destruction methods for prostate cancer using beta rays (Lu-PSMA therapy) or with alpha rays (Ac-PSMA therapy). There is no need for surgery, since tumor cells are destroyed via a non invasive manner, by electrocution using the fuel cell(s) coupled to the tumor cell specific ligand at the specific site to be treated.

The present invention will be further detailed in the following examples and figures wherein:

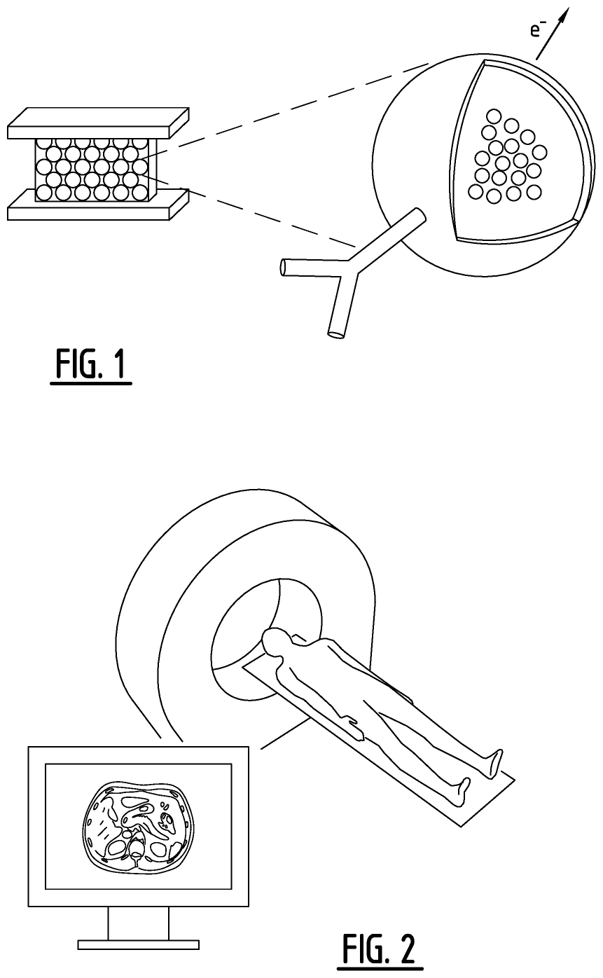

FIG. 1: shows a schematic of the nano-full cell coupled to at least one ligand for specific binding to a cell membrane, preferably of a tumor cell. The plates are representations of electrodes of which the upper is the cathode and the plate below is the anode. The hollow particles are represented as circles of which one is enlarged at the right of the picture. The hollow particle is composed a substrate permeable and electrically conductive outer polymer shell that can easily conduct electrons, and at the same time provides an entrapped vesicle for an enzyme inside said particle. The hollow particles contain an enzyme, preferably the enzyme glucose oxidase, that can convert glucose into gluconolactone, releasing electrons. Electrons are being released from the nano-fuel cell after contact with a substrate, for example glucose which is enzymatically processed, being the substrate of the enzyme glucose oxidase which is entrapped in the hollow particle or in another embodiment may be coated onto the electrodes of the nano-fuel cell. The at least one ligand (for example a specific antibody) can bind to a specific cell membrane protein, preferable of a tumor cell membrane protein.

FIG. 2: shows a patient having an IV infusion of the contrast agent of present invention administered during imaging, inside an MRI apparatus for detection of tumor cells inside the body.

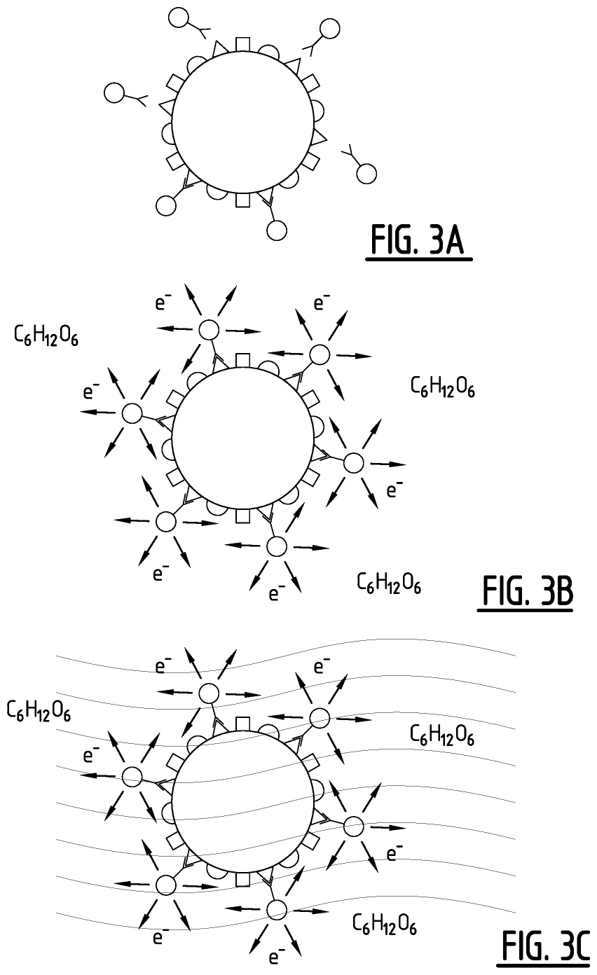

FIG. 3: shows the working principle of the contrast agent of the present invention. FIG. 3a shows a tumor cell having various cell receptors, of which one is tumor specific (indicated as a triangle). The contrast agent of present invention comprises a ligand for specific binding to this tumor cell. FIG. 3b shows that the contrast agent of present invention is activated by a substrate, e.g. by glucose which is enzymatically processed by glucose oxidase inside the nano-fuel cell, generating a flow of electrons along the cell membrane. FIG. 3C shows that when the contrast agent in activated by glucose and generates a flow of electron (current), during the MRI scan this generation of electrons is detected and indicates on the MRI image the specific presence and position of the tumor cells.

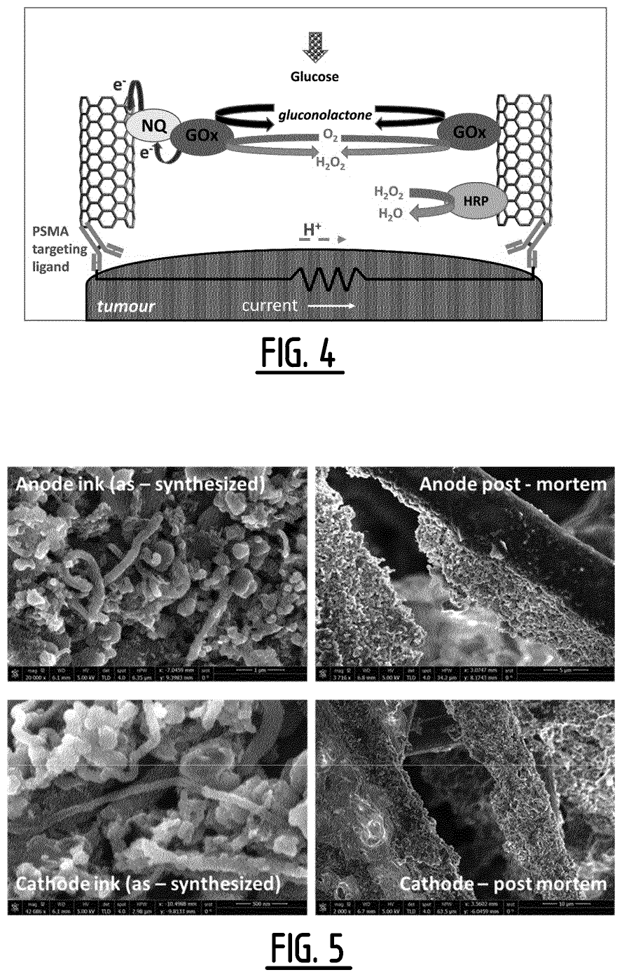

FIG. 4: Shows an overview of the design of the bio-anodes of the contrast agent focused on the use of glucose oxidase (GOx), while a bi-enzymatic approach is adopted for the bio-cathodes based on the combination of GOx and horseradish peroxidase (HRP). GOx was used as bio-anode using mediated electron transfer via 1,4-naphthoquinone (NQ). Both the cathode and the anode (left and right) utilise multiwall-walled carbon nanotube (MWCNT) as immobilization vehicles for coupling of the at least one ligand to the nano-fuel cell. The operation of the proposed nEGFC is illustrated; electrocatalytic oxidation of glucose at the bio-anode created by the simultaneous confinement of GOx and naphthoquinone (NQ) mediator on the MWCNT support. The bio-cathode operation is based on synergetic bi-enzymatic action—(i) production of H2O2 via O2 reduction by GOx via glucose oxidation and (ii) reduction of as produced H2O2 at low overpotentials by HRP directly wired on MWCNTs.

FIG. 5: Shows a scanning electron microscopy (SEM) image of the electrodes comprised of multiwall-walled carbon nanotubes MWCNT, before and after being coated with enzyme including GOx (anode), GOx and HRP (cathode).

### EXAMPLES

### Production of the MRI Contrast Agent Comprised of the Nano-Fuel Cell and Ligand

Encapsulation of the glucose oxidase of the GOx enzymes into the hollow particles was carried out by preparing a solution of 48 mg/l GOx dissolved in phosphate buffer (20 mM, pH 7.0). Into this solution a 1.0 mg/ml solution of polystyrene-b-poly (L-isocyanoalanine (2-thiophen-3-yl-ethyl)amide) (PS-PIAT) in Tetrahydrofuran (THF) was injected resulting in a final buffer to THF ratio of 6:1 (v/v). The free enzyme was removed by size exclusion chromatography using Sephadex G-50 and an aqueous phosphate buffer (pH 7.5) as eluent.

Next, Cross-linking of the PS-PIAT polymer membrane was done by making an aqueous solution of 0.20 ml of 30 mg/l Candida antarctica: lipase B (CAL B) and 1.0 ml of 1.3 μM bis (2,2′-bipyridine)ruthenium (II) bis (pyrazolyl) (BRP) in which 0.10 ml of a solution containing 0.50 g/l PS-PIAT in THF was injected, resulting in a final water/THF ratio of 12:1 (v/v). A concentration of BRP was chosen that was comparable to the amount of thiophene groups present (2×10⁻⁷ M). Subsequently, the dispersion was placed in a water bath of 60° C. for the desired period of time. After cooling to room temperature 0.50 ml of the dispersion was transferred to an Eppendorf having a filter unit with a cut-off of 100 kDa. The dispersion was centrifuged to dryness after which 0.50 ml of pure water was added and the dispersion was centrifuged again to dryness. After repeating this step a second time, 0.50 ml of water was added to redisperse the cross-linked aggregates.

A confined reaction chamber confined reaction chamber of about 1-2 cm³ is filled with a water-based dispersion of the Glucose Oxidase-containing vesicles. The ‘fuel’ glucose can be dissolved in this dispersion up to relatively high concentrations. Two electrodes (constructed of e.g. Indium Tin Oxide (ITO)) are attached on the top and the bottom of the reaction chamber and upon the application of a voltage, electrons generated in the fuel cell are be transported to an external capacitor from which a constant current is obtained.

Electrochemical Analysis on Nano-Enzymatic Glucose Fuel Cell (nEGFC) for In-Vivo Applications

A large number of nano-dimensional electrodes (anodes and cathodes) are delivered to the targeted tumour with intention to self-assemble on the its surface, forming statistically significant number of nano-Enzymatic Glucose Fuel Cells (nEGFCs), by intravenous administration into the blood stream. These nEGFCs can be selectively attached to a cancerous tissue via specific antibody/ligand conjugation, such as PSMA. In such a manner, the soft cancerous tissue serves as an electrical load for discharging the as formed nEGFCs. The resulting current enhances the MRI imaging resolution as well as play a therapeutic role (at sufficiently high currents).

In this experiment, the design of the bio-anodes is focused on the use of glucose oxidase (GOx), while a bi-enzymatic approach is adopted for the bio-cathodes based on the combination of GOx and horseradish peroxidase (HRP). GOx was used as bio-anode using mediated electron transfer via 1,4-naphthoquinone (NQ). Both the cathode and the anode utilise multiwall-walled carbon nanotube (MWCNT) as immobilization vehicles. The operation of the proposed nEGFC is illustrated in FIG. 4. Equation 1 below represents the electrocatalytic oxidation of glucose at the bio-anode created by the simultaneous confinement of GOx and naphthoquinone (NQ) mediator in the MWCNT support. The bio-cathode operation is based on synergetic bi-enzymatic action—(i) production of H₂O₂ via O₂ reduction by GOx via glucose oxidation (eq.2); (ii) reduction of as produced H₂O₂ at low overpotentials by HRP directly wired on MWCNTs (eq.3).

$$Anode : \\ Glucose \to Gluconolactone + 2 e - + 2 H + ( electrocatalytic oxidation of glucose ) \qquad ( 1 )$$

$$Cathode :$$

$$O 2 + 2 e - + 2 H + \to H 2 O 2 ( production of H 2 O 2 by GOx ) \qquad ( 2 )$$

$$H 2 O 2 + 2 e - + 2 H + \to 2 H 2 O ( electroenzymatic \\ reduction of H 2 O 2 by HRP ) \qquad ( 3 )$$

### Material & Methods

Both enzymes, HRP (1000 U mg-1) and GOx (220 U mg-1) were purchased from SERVA. The 1,4-naphthoquinone (NQ), D-(+) glucose and PBS buffer were purchased from Sigma Aldrich. The precursors were used as received. Multi-wall carbon nanotubes MWCNTs (>90% purity) were sources from research projects within the Department of Materials Science and Metallurgy, University of Cambridge.

The electrochemical experiments were carried out in a three-electrode electrochemical cell configuration using Bio Logic VNP3B-5 electrochemical workstation. A platinum wire was used as the counter electrode and Ag/AgCl served as reference electrode. The potentials given here are referred vs. Ag/AgCl electrode. All experiments were conducted in phosphate-buffered saline solutions at pH 7.4 (PBS: 0.02 M phosphate buffer) at ˜37 C.

The morphology of the MWCNT electrodes was investigated by high resolution FEI Nova Nano SEM. The nano-electrode ink suspensions were drop-casted into two carbon-felt electrodes {˜ 1 cm2 area). The electrodes were attached to gold current paths using Leit-C carbon paint (Sigma-Aldrich). The gold electrodes were sputtered in-house onto a glass substrate.

The electrodes were produced, briefly 30 milligrams of MWCNT (30 mg) were sonicated in 3 ml of ultra-pure water for 30 min. The resulting suspension was divided in two equal parts of 1.5 ml and stirred magnetically for further 30 min at 1500 rpm. GOx (3 mg) and NQ (1.5 mg) were added to the anode ink. HRP (3 mg) and GOx (0.3 mg) were added to the cathode ink. After 1 h of stirring, the suspensions were transferred into separate micro-centrifuge tubes and centrifuged at 5000 rpm for 10 min. Then 100 μL of the supernatant was drop-cased on each of the two carbon-felt electrodes. The electrodes were dried on a hot plate at 40° C. for 30 min, resulting in a GOx/NQ anode and a HRP+GOx cathode (See FIG. 5). Both bio-electrodes were tested at pH 7 in the presence of 10 mM of glucose solution in PBS buffer.

The Enzymatic Glucose fuel cell (EGFC), is coupled to a ligand, more specifically PSMA for use in MRI imaging i.e. a PSMA-PET-CT. The nano fuel cell is comprised of single of multi walled carbon nanotubes (SWCNTs or MWNTs), to which the PSMA ligand (pharmacophore) is linked as follows: SWNTs are treated with (4-pyrenyl)butanoyl-PEG-Lys-CO-glu before the co immobilization of glucose oxidase (GOx) and horseradish peroxidase (HRP) is carried out. The amount of the (4-pyrenyl)butanoyl-PEG-Lys-CO-glu must be optimized so that the immobilized PSMA ligand does not prevent the immobilization of the redox enzymes and sufficient binding affinity to the target is maintained.

Alternatively, already assembled electrodes are treated with amine reactive TfpO2C-PEG-Lys-CO-Glu. The ligand conjugation was optimized so that it does not significantly interfere with the enzymatic activity of GOx and HRP but affords an adequate number of PSMA binding sites.

### Results

The nano biofuel cell was then characterized by variable load discharge through an external variable resistor (10 to 0.5 kΩ). Note, that the resistive values of tumour's tissue were reported in the literature to vary between ˜100Ω to few kΩ. An open circuit voltage (OCV) of 370-390 mV was observed initially after the stabilisation for ˜1.5 hours. The discharge under variable load registered currents ranging from 20 μA to 6 μA. The OCV dropped to ˜250 mV after an overnight discharge in 10 mM glucose solution. Such drop in OCV voltage of ˜30% was accompanied by slight obfuscation of the glucose solution evidencing a partial leaching of the inks.

Next, cyclic voltammetry (CV) experiments were carried out at slow scan rate (1 mV/s) in order to avoid capacitive contribution from the experimental set-up. Cyclic voltammetry is used to study the electrochemical properties of the electrodes. The CV in three-electrode configuration was used to evaluate the bio-electrocatalytic current densities for glucose oxidation by anodes and oxygen reduction by cathodes after an overnight operation. Slow-scan CV responses of the anode (GOx/NQ) and cathode (HRP+GOx) inks immobilised on MWCNTs in the presence of 0.1 mM glucose solution at pH 7.4 were monitored. Maximum steady-state current density of ˜970 μAcm-2 is observed for the anode. The highest steady-state current density of ˜370 μAcm-2 was observed for oxygen reduction by the bi-enzymatic cathode.

Scientific papers disclose the detection of electric field fluctuations through neurons by MRI. The ionic currents with current densities on the same order of magnitude as those induced by neuroelectric activity in nerve fibers can be detected by MRI. Truong et al. 2006, Journal of Magnetic reason, March; 179(1) 85-91 discloses that the lower limit of MRI detection is about 1-10 microA, and that the current generated by a single neuron is on the order of nanoamperes depending on its diameter, typically 104 to 105 neurons/mm, and can generate a current density in the order of tens of μAmm-2. This would be compatible with the nano-Enzymatic Glucose Fuel Cell (nEGFC) at the applied magnetic field strength in an MRI, producing at least 300 μAcm-2.

1. A contrast agent for MRI imaging diagnostics comprised of a nano fuel cell coupled to at least one ligand for specific binding to a cell membrane,
wherein the nano fuel cell is comprised of an anode compartment including an anode, a cathode compartment including a cathode, and a suspension for generating a current of electrons,
wherein said suspension is preferably comprised of a plurality of hollow particles in electrically conductive contact,
wherein said hollow particles comprise entrapped therein a redox-reaction for catalyzing an enzymatic conversion of a substrate in said hollow particles thereby liberating electrons, and
wherein said hollow particles comprise a substrate permeable and electrically conductive outer polymer shell, and said suspension is disposed within said anode compartment, or within said cathode compartment, or between said anode and cathode compartment.

2. The contrast agent according to claim 1, wherein the at least one ligand is one or more selected from the group consisting of affinity binding molecules, antibodies, mini-bodies, FAB fragments, ligand proteins, peptides, RNAs, small molecules and nano-particles.

3. The contrast agent according to claim 1, wherein the at least one ligand is able to bind to a specific cell membrane protein, selected from the group consisting of a tumor cell membrane protein, PSMA, FAP, CAIX, SSTR, αvβ3 integrin, Bombesin R, CEA, CD13, CD44, CXCR4, EGFR, ErbB-2, Her2, Emmprin, Endoglin, EpCAM, EphA2, Folate, GRP78, IGF, Matriptase, Mesothelin, cMET/HGFR, MT1-MMP, MT6-MMP, Muc-1, PSCA, Tn antigen and uPAR, preferably PSMA, FAP, CAIX, and/or SSTR.

4. The contrast agent according to claim 1, wherein the at least one ligand is furthermore conjugated with radionuclides and/or near-infrared fluorescent dyes selected from the group consisting of beta particles emitting isotopes, alpha particles emitting isotopes, auger electrons emitting isotopes, fluorophores, F-18, Ga-68, Zr-89, Cu64, I-124, Tc-99m, In-111, and I-123.

5. The contrast agent according to claim 1, wherein the polymer of said outer polymer shell is a block-copolymer comprising a hydrophobic polystyrene and a hydrophilic polyisocyanopeptide.

6. The contrast agent according to claim 1, wherein said redox-reaction catalyzing enzyme is glucose oxidase (GOx), preferably GOx in combination with horseradish peroxidase (HRP), and said substrate is glucose.

7. The contrast agent according to claim 1, wherein the cathode and/or anode are enzyme-coated with redox-reaction catalyzing enzyme(s), preferably the cathode and/or anode are coated with glucose oxidase (GOx), more preferably GOx in combination with horseradish peroxidase (HRP).

8. The contrast agent according to claim 1, wherein the nano fuel cell further comprises Naphthoquinone (NQ).

9. The contrast agent according to claim 1, wherein the hollow particles are embedded in an electrically conductive matrix comprised of ferrocene and/or viologen derivatives.

10. The contrast agent according to claim 1, wherein the contrast agent is an MRI imaging contrast agent.

11. The contrast agent according to claim 1, wherein said contrast agent is furthermore suitable for medical imaging diagnostics selected from the group consisting of CT, PET and SPECT.

12. The contrast agent according to claim 1, wherein said nano-fuel cell coupled to at least one ligand has a particle size of approximately 20 to 400 nm, preferably 50 to 250 nm, more preferably 75 to 200 nm.

13. The contrast agent according to claim 1, wherein the cathode and the anode are comprised of single or multiwall-walled carbon nanotubes (SWCNT or MWCNT) and/or metal nanoparticles for coupling of the at least one ligand to the nano-fuel cell.

14. The contrast agent according to claim 1, wherein the at least one ligand comprises pyrenyl residues for stable non-covalent coupling of said ligand to the nano-fuel cell.

15. The contrast agent according to claim 1, wherein the outer polymer shell is coated with metal particles selected from the group consisting of Ag, Au, Pt, Ti, preferably Au.

16. An imaging method for the visualisation of tumor cells comprising the steps of;
a) providing contrast agent according to claim 1 to a patient,
b) providing glucose to said patient for providing activation of the contrast agent and generating a current of electrons that is detectable by medical imaging techniques,
c) obtaining an image of the contrast agent using medical imaging techniques

17. The imaging method according to claim 16, wherein the medical imaging technique selected from the group consisting of MRI, Magnetometric imaging (MEG), CT, PET and SPECT, preferably MRI.

18. The imaging method according to claim 16, wherein said contrast agent is detectable by imaging diagnostics for 1 to 90 minutes, after providing said contrast agent to said patient.

19. A method for non-invasive treatment of tumor cells or macrophages comprising the steps of;
a) providing contrast agent according to claim 1 to a patient, and
b) providing an overdose of glucose to said patient resulting in electrocution and/or electroporation of the tumor cells or macrophages.
